(19) United States
(12) Patent Application Publication
(10) Pub. No.: US 2018/0292024 A1
(43) Pub. Date: Oct. 11, 2018

(54) EXTERNAL PEDAL FOR ATTENUATION AND LOCKING INTERRUPTION OF THE FLOW OF SUCTION

(71) Applicant: BOARD OF REGENTS, THE UNIVERSITY OF TEXAS SYSTEM, Austin, TX (US)

(72) Inventors: Michael Clark, Dallas, TX (US); Dustin Buller, Dallas, TX (US); Katherine Krenek, Dallas, TX (US); Dietrich Riepen, Dallas, TX (US); Lindsey West, Dallas, TX (US)

(21) Appl. No.: 15/950,500

(22) Filed: Apr. 11, 2018

Related U.S. Application Data
(60) Provisional application No. 62/484,237, filed on Apr. 11, 2017.

(51) Int. Cl. F16K 31/62; A61M 1/00; A61C 1/00; A61C 17/06; F16K 7/06; F04B 49/22

(52) U.S. Cl. CPC F16K 31/62; A61M 1/0035; A61C 1/0023; A61M 2210/1025; F16K 7/066; F04B 49/225; A61M 2205/582; A61C 17/043

(57) ABSTRACT

The present invention includes an apparatus and method for a hands-free foot pedal to attenuate and interrupt suction that attaches externally around suction tubing, in the context of any system where the flow of suction is through tubing.

### CROSS-REFERENCE TO RELATED APPLICATIONS

This patent application claims priority to U.S. provisional patent application Ser. No. 62/484,237 filed on Apr. 11, 2017 entitled “External Pedal for Attenuation and Locking Interruption of The Flow of Suction,” all of which is hereby incorporated by reference in its entirety.

### TECHNICAL FIELD OF THE INVENTION

The present invention relates in general to the field of surgical assist devices, and more particularly, to an external pedal for attenuation and locking interruption of the flow of suction.

### STATEMENT OF FEDERALLY FUNDED RESEARCH

Not Applicable.

### INCORPORATION-BY-REFERENCE OF MATERIALS FILED ON COMPACT DISC

Not Applicable.

### BACKGROUND OF THE INVENTION

Without limiting the scope of the invention, its background is described in connection with suction modulation devices.

Previous devices have outlined solutions to modulate suction, but are inadequate to solve many of the problems mentioned above. U.S. Pat. No. 4,702,733 describes a hand-held irrigation and aspiration system in which the foot pedal actually serves to selectively increase the aspiration force. U.S. Pat. No. 4,696,669 also describes a device to modulate suction, however this apparatus is hand-held and connected in series via valves with a constant vacuum source. While these devices both modulate suction intensity, both do so as part of a larger system, including the suction itself either internally or connected in series.

In particular, U.S. Pat. No. 4,702,733 is useful primarily in the setting of clinical dentistry, where suction is not centrally located and controlled. However, in many of the systems with which we are concerned suction is centrally located, with a valve control located on or within one of the walls of a room. Additionally, U.S. Pat. No. 4,720,733 contains its own internal vacuum reservoir, which, given its reported dimensions, may be much too small for the amount of suctioned debris which can accumulate over the course of a surgical operation, especially as compared to the central suction reservoirs with which most hospitals are equipped. Thus U.S. Pat. No. 4,702,733 does not provide a solution for the external modulation and/or interruption of the pre-existing suction in these settings.

Similarly, U.S. Pat. No. 4,696,669 includes its own internal, motor-generated suction system and reservoir. As with U.S. Pat. No. 4,702,733 the suction system and reservoir are redundant in the setting of a science laboratory, operating room, or emergency room where there is a pre-existing central suction system.

### SUMMARY OF THE INVENTION

In one embodiment, the present invention includes a device comprising: a hands-free foot pedal that attenuates and interrupts a suction, wherein the foot pedal attaches externally around a suction tubing, wherein a flow of the suction is through the tubing. In one aspect, the foot pedal further comprises a locking mechanism that allow for complete and indefinite interruption of suction without continued applied force by an operator on the foot pedal. In another aspect, the foot pedal further comprises a three-point pinching mechanism that allows modulation of suction intensity as the pedal is depressed. In another aspect, the foot pedal further comprises an elliptical flat spring below the center pinching point that allows for increased user resolution, as defined by angle of pedal depression, in the attenuation of suction. In another aspect, the foot pedal further comprises a double hinge allowing for a hinge with resistance from the neutral position to the closed and locked position, and another hinge allowing for opening of the pedal from the neutral to the open position. In another aspect, the foot pedal further comprises maintains the neutral position with a magnetic interaction. In another aspect, the foot pedal can be opened easily from neutral position to allow access to the tubing without tools. In another aspect, the foot pedal further comprises a ribbed retaining clip that accommodates variable diameters of the tubing by pressing the tubing into the ribbed retaining clip. In another aspect, the foot pedal further comprises one or more anti-slip ribs, each of the one or more ribs comprising a unique pedal identifier, wherein the pedal identifier allows for tactile discrimination of the one or more anti-slip ribs or the one or more varying surfaces of the foot pedal.

In another embodiment, the present invention includes a method for hands free control of a suction comprising: providing one or more a hands-free foot pedals that attenuate and interrupt a suction from one or more sources, wherein the foot pedal attaches externally around a suction tubing, and depressing the foot pedal to control a flow of the suction through the tubing.

A hands-free foot pedal to attenuate and interrupt suction that attaches externally around suction tubing, in the context of any system where the flow of suction is through tubing.

In another embodiment, the present invention includes a foot pedal with a locking mechanism to allow for complete and indefinite interruption of suction without continued applied force by the operator. In another embodiment, the present invention includes a foot pedal with a three-point pinching mechanism that allows modulation of suction intensity as the pedal is depressed. In another embodiment, the present invention includes a foot pedal with an elliptical flat spring below the center pinching point that allows for increased user resolution, as defined by angle of pedal depression, in the attenuation of suction. In another embodiment, the present invention includes a foot pedal with a double hinge allowing for a hinge with resistance from the neutral position to the closed and locked position, and another hinge allowing for opening of the pedal from the neutral to the open position. In another embodiment, the present invention includes a foot pedal that maintains the neutral position with magnetic interaction. In another embodiment, the present invention includes a foot pedal that can be opened easily from neutral position to allow access to the tubing without tools. In another embodiment, the present invention includes a ribbed retaining clip to accommodate variable diameters of tubing simply by pressing the tubing into it. In another embodiment, the present invention includes a foot pedal with varying anti-slip ribs to allow for pedal identification as well as tactile discrimination of the varying surfaces of the pedal.

### BRIEF DESCRIPTION OF THE DRAWINGS

For a more complete understanding of the features and advantages of the present invention, reference is now made to the detailed description of the invention along with the accompanying figures and in which:

FIG. 1 is an isometric view of the neutral position.

FIG. 2 is a side view of the neutral position of the pedal.

FIG. 3 is an isometric view of the open position of the pedal.

FIG. 4 is a transverse cross-sectional view of the closed and locked pedal with a view of the suction tubing in place.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

What follows is a thorough description of the above figures that utilizes reference numbers, along with a discussion of their function in attenuating or interrupting surgical suction. This description and discussion is not intended to limit the scope of application of this invention.

Part names and numbers for reference in the figures:

1. Upper Pedal

2. Base

3. Ribbed Retaining Clip

4. Upper Latch

5. Lower Latch

6. Pedal Latch Release

7. Elliptical Flat Spring

8. Base Torsional Springs

9. Center Tubing Compression Wedge

10. Right Tubing Compression Wedge

11. Left Tubing Compression Wedge

12. Upper Latch Clearance Slot

13. Base Friction Grommet

14. Upper Pedal Anti-Slip Ribs

15. Pedal Latch Release Anti-Slip Ribs

16. Base Hinge

17. Intermediate Hinge Piece

18. Upper Pedal Hinge

19. Pedal Latch Release Hinge

20. Pedal Retention Magnets

21. Pedal Latch Release Torsion Springs

22. Base Hinge Projections

23. Suction Tubing

An external, hands-free foot pedal is here disclosed, for both the attenuation and complete interruption of the flow of suction. The device is intended for use in any suction application, particularly situations where control over suction intensity is desired, such as in healthcare settings like the operating room. Suction tubing is placed into a retaining clip within the pedal. As the pedal is depressed by the operator's foot, the suction tubing is pinched at three locations simultaneously, resulting in a reduction in suction intensity at the tip of the suction handle. When the pedal is fully depressed by the operator, suction flow is completely interrupted. There is also a locking mechanism for maintaining the pedal in the fully depressed state indefinitely without any continued pressure to the foot pedal by the operator. This can be unlocked by foot when the operator desires to restore the flow of suction. This foot pedal can be applied to any system in which suction flows through tubing by connecting around it externally, without any other machinery that must be connected in series with, or is internal to, the device itself.

There are many applications for suction in the setting of the healthcare and science industries. Suction can be used to remove secretions, blood, and saliva from the airway to facilitate breathing, as well as for visualization in dental settings. In surgeries, suction is used to allow for better visualization of the surgical field and for removal of unwanted substances like blood, pus or tissue. Because some surgical sites are small and delicate, such as those in ENT and pediatric surgeries, it would be beneficial to have the ability to both attenuate the strength of suction through tubing and to completely switch off suction when desired.

Aside from utility in the setting of small or delicate anatomical areas, greater control of surgical suction yields a variety of other possible intra-operative benefits. Some surgeons suggest that improved control of suction intensity allows for better manipulation of tissue and grafts during operative procedures. In addition, better control of suction may lead to reduced time in the operating room, which could lead to decreased incidence of infection, as one of the primary risk factors for surgical site infections is increased length of operation. Furthermore, histologic studies have demonstrated that excessive suction can damage tissue and thus possibly impair wound healing. The present invention offers a solution, in part, to these problems.

An additional benefit of this device's ability to interrupt suction is the reduction of noise in in the operating room or other healthcare settings. The noise of constant suction contributes to distraction and inhibited communication amongst healthcare personnel.

Furthermore, it is sometimes the case during an operative procedure that the suction tip catches a piece of draping and creates a loud, high-pitched noise. The ability to quickly and easily control the flow of suction with a foot pedal can help alleviate these noise-related issues.

Typically, control of suction in the operating room or other healthcare setting is performed in an all-or-none fashion. To interrupt suction, the surgeon or an assistant usually uses an operative tool to manually clamp the tubing closed. If a lower suction intensity is desired, the surgeon must communicate to other operating room staff to adjust the intensity at the suction source, which is usually from a central or “wall” source. This takes time, requires increased communication, and necessitates that staff are distracted from other tasks.

In the present device an apparatus and methods are provided by which one is able to both attenuate and completely interrupt the suction of any external suction system in which tubing is employed for the delivery of suction forces and debris. Examples of where such suction systems are employed include, but are not limited to, scientific laboratories, operating rooms, emergency rooms, and various other scientific and healthcare settings. The device is particularly adapted for use with surgical suction systems, and so will be described in this context by way of illustration, though its use is by no means limited to the operating room.

The device is a pedal that provides operators with a hands-free means to both attenuate and completely interrupt suction, external to any existing suction system.

It includes a locking mechanism that allows for indefinite, complete interruption of suction without continued application of any force by the operator. The device also utilizes magnets that maintain the pedal in the neutral position and provide an easy way for the operator to open the pedal without the use of tools.

Attenuation and interruption of suction is accomplished by pinching the suction tubing between three vertically oriented wedges within the pedal. The tubing is first placed firmly within a v-shaped ribbed retaining clip and then runs between the lower base plate and upper modulating plate of the pedal. The ribbed retaining clip accommodates various sizes of tubing. At the center of the base plate, the tubing rests on a center tubing compression wedge attached to an elliptical linear spring that is able to be compressed downwards with the depression of the pedal. The other two tubing compression wedges protrude downwards from the modulating upper pedal plate on both sides the elliptical spring and compress the tubing from above when the pedal is compressed by the operator. Thus, there are a total of three pinch points on the tubing. If the operator partially compresses the pedal, suction can be modulated to varying degrees. A number of resistance systems have been incorporated into the pedal to allow for finer control of pedal depression and tube compression by the operator.

The locking mechanism mentioned above consists of a catch that serves to lock the pedal in the closed position and interrupt suction indefinitely. This can be reversed by a pressing downward on the front edge of the pedal, which will release the catch, allowing for the return of the pedal to neutral position.

Finally, the upper surface of the pedal contains a number of raised anti-slip ribs, which serve as a means for the operator to (1) identify the pedal, (2) differentiate between the front “unlocking” end of the pedal and the rear “compressing” end, and (3) provide increased friction when gripping the pedal by foot.

Various objects, features, aspects, and advantages of the present inventions will become more apparent from the following detailed description and drawings.

Thus, there is still a great need to provide an apparatus and method for the external attenuation and interruption of the pre-existing suction systems, which are present in the aforementioned settings and others like them.

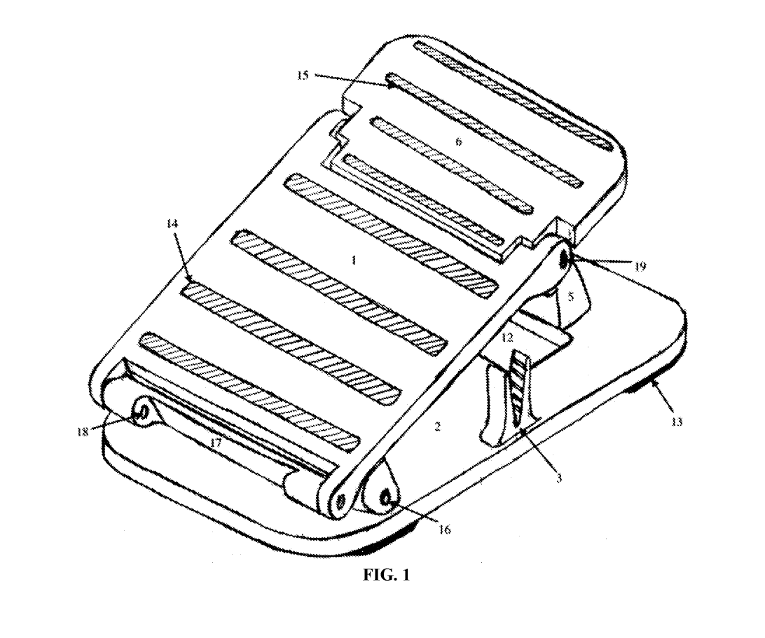

FIG. 1 is an isometric view of the neutral position. On the top of the upper pedal (1), there are upper pedal anti-slip ribs (14). These are linear, horizontal protrusions on the superior surface of the pedal that help the operator identify and grip the pedal with his or her feet. More distally there is the pedal latch release (6) with pedal latch release anti-slip ribs (15) that are thicker, more raised, and at shorter intervals when compared to the upper pedal anti-slip ribs (14). The difference in thickness, height, and spacing serves as tactile discrimination between the upper pedal (1) and the more distal pedal latch release (6).

Also visible from this view are the base hinge (16), intermediate hinge piece (17), and upper pedal hinge (18). These allow for depression of the pedal and resultant compression of the suction tubing. In addition, the latch release hinge (19) is visible in this view and functions in releasing the locking mechanism.

Underneath the base (2), there are four rubber base friction grommets (13) to stabilize the base (2) of the pedal and prevent slippage when pressure is applied to the upper pedal (1). Two of the base friction grommets (13) are visible in FIG. 1.

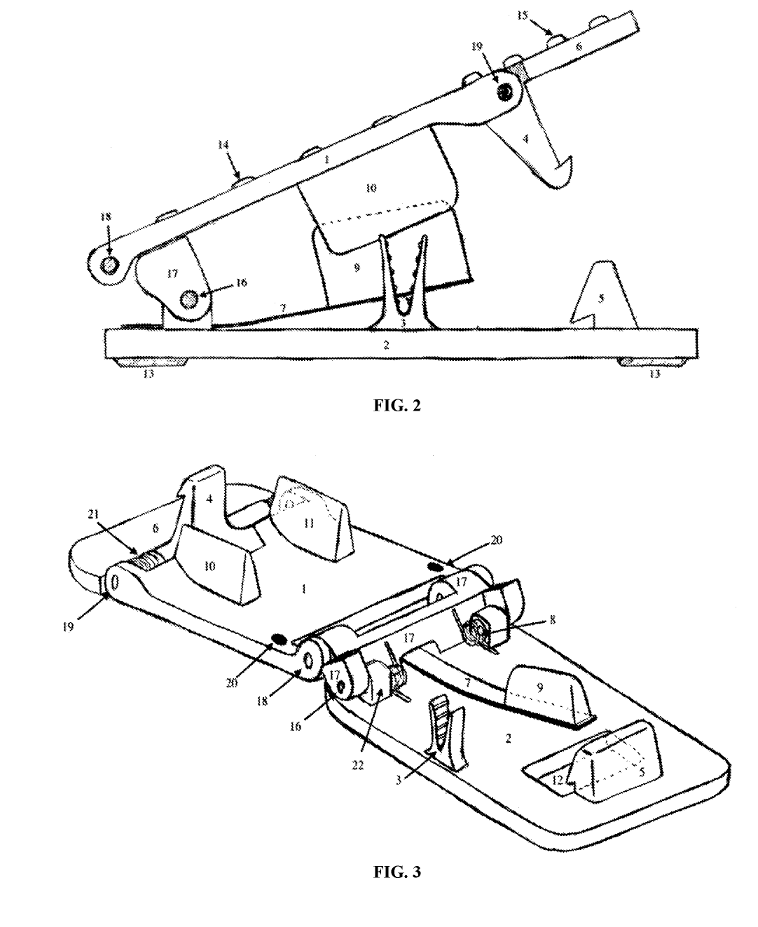

Other features that are visible in FIG. 1 but not discussed here will be discussed in FIGS. 2-4 where they are more plainly seen.

FIG. 2 is a side view of the neutral position of the pedal. Again visible are the upper pedal (1) and base (2). On the proximal side of the base (2) from this perspective, there is a ribbed retaining clip (3) that secures the suction tubing (not shown) in place.

An elliptical flat spring (7) extends axially down the length of the base and angles superiorly off of the base. A center tubing compression wedge (9) is attached to the superior end of the elliptical flat spring (7). The tubing of an external suction system runs through the V-shaped ribbed retaining clip (3), which allows for accommodation of varying diameters of tubing. It then runs over the center tubing compression wedge (9) and across the base (2) to the side opposite the V-shaped ribbed retaining clip. There are right and left tubing compression wedges (10, 11) protruding from the inferior surface of the upper pedal (1) that serve as two lateral pinch points on the tubing when the pedal is compressed. In total there are three pinch points: the right and left tubing compression wedges (10, 11) descending from the upper pedal (1) and the center tubing compression wedge (9) attached to the elliptical flat spring (7).

In this neutral position, the tubing is in proximity to the central, right, and left compression wedges (9, 10, 11) but is not compressed. With increasing depression of the upper pedal (1), the tubing is kinked by the three compression wedges (9, 10, 11) and suction intensity is attenuated.

Towards the front (the right side of this drawing) of the upper pedal (1) and base (2), there are upper and lower latches (4, 5) that are angled towards each other and slide against each other as the upper pedal (1) is depressed. This contact causes automatic downward rotation of the pedal latch release (6), such that when the upper pedal (1) is fully compressed, the upper and lower latches (4, 5) will automatically lock. These latches (4, 5) secure the pedal in the fully compressed and closed position, leaving the flow of suction indefinitely and completely interrupted. When pressure is applied to the pedal latch release (6), it pivots on the pedal latch release hinge (19) with torsion springs (21). The upper latch (4) can be released when the pedal latch release (6) is compressed on the upper pedal (1), allowing for restoration of suction flow.

FIG. 3 illustrates the pedal in the open configuration. In the open configuration, the base torsional springs (8) are visible. These base torsional springs (8) resist depression of the pedal through their connection between intermediate hinge piece (17) and the base (2). The base hinge projections (22) hold the hinge between the intermediate hinge piece (17) and the base (2), providing the axis for the base torsional springs (8). The tubing (not shown here) is secured into place in the ribbed retaining clip (3) and runs over the center tubing compression wedge (9). Both right and left tubing compression wedges (10, 11) are visible on the inferior side of the upper pedal (1).

When the pedal is closed, the pedal retention magnets (20) hold the upper pedal (1) in place by connecting to another set of magnets (not pictured) that are flush with the superior surface within the intermediate hinge piece (17). The pedal retention magnets (20) are visible on inferior surfaces of the upper pedal near the upper pedal hinge (18).

The open configuration illustrates the locking mechanism in greater detail. Adjacent to the lower latch (5) on the base (2) is an upper latch clearance slot (12). This slot allows the upper latch (4) extra distance to extend below the lower latch (5). When the upper pedal (1) is compressed, the upper latch (4) contacts the lower latch (5), leading to downward rotation of the pedal latch release (6). This causes the pedal to automatically lock when fully compressed as the upper latch (4) extends into the upper latch clearance slot (12) and locks with the lower latch (5).

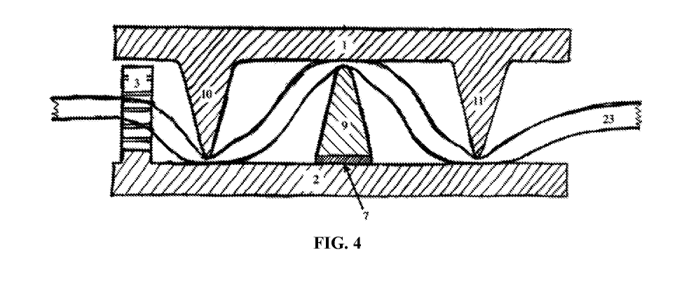

In FIG. 4, which shows the transverse cross sectional view of the fully compressed and locked configuration, the lumen of the external tubing (23) is obliterated and flow of suction ceases. The locking mechanism (via the upper and lower latch, not shown here) maintains the locked state indefinitely. To restore suction flow, the operator depresses the pedal latch release (6) to disengage the locking mechanism. When the pedal is fully compressed, the tubing (23) is pinched by the center, right and left tubing compression wedges (9, 10, 11).

As the pedal is initially depressed from the neutral position, a center pinch point is created between upper pedal (1) and the center tubing compression wedge (9). As this center pinch point begins to compress the tubing (23), pressure is also transmitted to the underlying elliptical flat spring (7), which is initially angled upward. When the pedal is fully depressed into the locked position, the elliptical flat spring (7) is compressed from its baseline angle of elevation in the neutral position to 0 degrees, as shown here, lying flat on the base (2).

This compression of the elliptical flat spring (7), when combined with the simultaneous compression of the tubing (23) between the center tubing compression wedge (9) and upper pedal (1), allows for a greater differential between the distance the pedal is depressed and the compression force applied to the tubing. This system affords the operator greater control over the attenuation of the suction by increasing the input angle required to compress the tubing.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention.

Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A device comprising:
a hands-free foot pedal that attenuates and interrupts a suction through a suction tube, wherein a pedal of the hands-free foot pedal attaches externally around the suction tube, wherein a flow of the suction is through the suction tube, and the pedal is connected to a base by a hinge, wherein the suction tube is capable of being connected to a vacuum source.

2. The device of claim 1, wherein the hands-free foot pedal further comprises a locking mechanism between the pedal and the base that allows for complete and indefinite interruption of suction without continued applied force by an operator on the hands-free foot pedal.

3. The device of claim 1, wherein the hands-free foot pedal further comprises a three-point pinching mechanism between the pedal and the base that allows modulation of suction intensity as the pedal is depressed.

4. The device of claim 1, wherein the pedal further comprises an elliptical flat spring positioned below a center pinching point of the pedal that allows for increased user resolution of the suction through the suction tube, as defined by angle of pedal depression, in an attenuation of suction.

5. The device of claim 1, wherein the hinge is defined further as a double hinge mechanism that allows for a first hinge with resistance from the neutral position to a closed and a locked position, and a second hinge that allows for opening of the pedal from the neutral to an open position.

6. The device of claim 5, wherein the pedal further maintains the neutral position with a magnetic interaction.

7. The device of claim 1, wherein the pedal can be opened easily from a neutral position to allow access to the suction tube without tools.

8. The device of claim 1, wherein the foot pedal further comprises a ribbed retaining clip below the pedal that accommodates variable diameters of the tubing by pressing the tubing into the ribbed retaining clip.

9. The device of claim 1, wherein the foot pedal further comprises one or more anti-slip ribs on an upper surface of the pedal, each of the one or more ribs comprising a unique pedal identifier, wherein the pedal identifier allows for tactile discrimination of the one or more anti-slip ribs or the one or more varying surfaces of the foot pedal.

10. A method for hands free control of a suction comprising:
providing one or more a hands-free foot pedals that attenuate or interrupt a suction from one or more vacuum sources, wherein the foot pedal attaches externally around a suction tubing and is attached by a hinge to a base, and
depressing the foot pedal to control a flow of the suction through the tubing.

11. The method of claim 10, wherein the foot pedal further comprises a locking mechanism that allow for complete and indefinite interruption of suction without continued applied force by an operator on the foot pedal.

12. The method of claim 10, wherein the foot pedal further comprises a three-point pinching mechanism that allows modulation of suction intensity as the pedal is depressed.

13. The method of claim 10, wherein the foot pedal further comprises an elliptical flat spring below the center pinching point that allows for increased user resolution, as defined by angle of pedal depression, in the attenuation of suction.

14. The method of claim 10, wherein the foot pedal is further defined as comprising a double hinge that allows for a first hinge with resistance from a neutral position to a closed and locked position, and a second hinge that allows for opening of the pedal from the neutral to an open position.

15. The method of claim 14, wherein the foot pedal further maintains the neutral position with a magnetic interaction.

16. The method of claim 10, wherein the foot pedal can be opened easily from a neutral position to allow access to the tubing without tools.

17. The method of claim 10, wherein the foot pedal further comprises a ribbed retaining clip below the foot pedal that accommodates variable diameters of the suction tubing by pressing the suction tubing into the ribbed retaining clip.

18. The method of claim 10, wherein the foot pedal further comprises one or more anti-slip ribs, each of the one or more ribs comprising a unique pedal identifier, wherein the pedal identifier allows for tactile discrimination of the one or more anti-slip ribs or one or more varying surfaces of the foot pedal.

19. A hands-free foot pedal capable of attenuating or interrupting a suction that attaches externally around suction tubing, wherein a flow of suction is through tubing, and the foot pedal is attached to a base by a hinge, wherein pressing the foot pedal compresses the suction tubing.

20. The foot pedal of claim 19, wherein a locking mechanism allows for complete and indefinite interruption of suction without continued applied force by an operator.

21. The foot pedal of claim 19, wherein a three-point pinching mechanism that allows modulation of suction intensity as the pedal is depressed.

22. The foot pedal of claim 19, wherein an elliptical flat spring below the center pinching point that allows for increased user resolution, as defined by angle of pedal depression, in the attenuation of suction.

23. The foot pedal of claim 19, wherein the hinge is defined further as a double hinge allows for a first hinge with resistance from a neutral position to a closed and locked position, and a second hinge allows for opening of the pedal from the neutral to the open position.

24. The foot pedal of claim 19, wherein the foot pedal maintains a neutral position by magnetic interaction.

25. The foot pedal of claim 19, wherein the foot pedal that can be opened easily from a neutral position to allow access to the tubing without tools.

26. The foot pedal of claim 19, wherein a ribbed retaining clip is provided to accommodate variable diameters of tubing simply by pressing the tubing into it.

27. The foot pedal of claim 19, wherein the foot pedal comprises varying anti-slip ribs to allow for pedal identification as well as tactile discrimination of the varying surfaces of the pedal.
